(19) United States
(12) Patent Application Publication
(10) Pub. No.: US 2025/0099046 A1
(43) Pub. Date: Mar. 27, 2025

(54) COMPUTED TOMOGRAPHY SYSTEM AND METHOD FOR ORIENTING A DETECTOR RING IN OR ON A GANTRY OF A COMPUTED TOMOGRAPHY SYSTEM

(71) Applicant: Siemens Healthineers AG, Forchheim (DE)

(72) Inventors: Hannes MOENIUS, Forchheim (DE); Hans-Juergen MUELLER, Pretzfeld (DE)

(73) Assignee: Siemens Healthineers AG, Forchheim (DE)

(21) Appl. No.: 18/894,216

(22) Filed: Sep. 24, 2024

(30) Foreign Application Priority Data
Sep. 27, 2023 (DE) 10 2023 209 449.5

(51) Int. Cl. A61B 6/03; A61B 6/00; A61B 6/06; A61B 6/42

(52) U.S. Cl. CPC A61B 6/035; A61B 6/06; A61B 6/4283; A61B 6/4435; A61B 6/4476; A61B 6/547

(57) ABSTRACT

A computed tomography system comprises: an examination region and a static gantry, wherein the gantry includes an X-ray source and a detector ring. The detector ring encircles the examination region and includes at least two detector segments. The computed tomography system is configured such that a position of the at least two detector segments relative to one another in an axial direction of the detector ring is adjustable.

### CROSS-REFERENCE TO RELATED APPLICATION(S)

The present application claims priority under 35 U.S.C. § 119 to German Patent Application No. 10 2023 209 449.5, filed Sep. 27, 2023, the entire contents of which is incorporated herein by reference.

### FIELD

One or more embodiments of the present invention relate to a computed tomography system and a method for orienting a detector ring in or on a gantry of a computed tomography system.

### BACKGROUND

Independent of the grammatical term usage, individuals with male, female or other gender identities are included within the term.

In the prior art, a detector of a computed tomography system (CT system) is typically arranged in or on a gantry of the CT system. The gantry is usually a short ring-shaped tunnel in the interior of which a subject, in particular a patient, or a part of the subject is placed for examination purposes. The detector generally comprises electronic components which are used for converting detected X-rays emitted by an X-ray tube into electronic signals. According to the prior art, the detector or a mechanism on which the electronic components of the detector are placed is generally designed as a partial fan (for example over) 45° and during a CT measurement is rotated together with the X-ray tube around the subject.

A precise orientation and fixing in place of the detector is important in this case for obtaining good CT image data. Owing to the rotation and the forces occurring as a result, the gantry and the mechanism responsible for the rotation must be implemented in a very stable design and therefore have a correspondingly high weight. The high weight limits the options for using the CT system, for example usually an exclusively stationary deployment is possible.

### SUMMARY

It is an object of one or more embodiments of the present invention to provide a computed tomography system, which can be used in a flexible manner, in particular as a result of a lower weight and/or by reducing or avoiding rotational forces. A mechanism and/or means for precise and/or as simple as possible orientation of the detector of the computed tomography system is also desirable.

At least this object is achieved via a computed tomography system and method according to the independent claims(s). Further features and advantages will become apparent from the dependent claims, the description and the attached figures.

According to a first aspect of an embodiment of the present invention, a computed tomography system is provided comprising an examination region and a static gantry comprising a detector ring which encircles the examination region. The detector ring comprises at least two detector segments, the computed tomography system being configured in such a way that a position of the detector segments in an axial direction of the detector ring can be adjusted. Advantageously, the computed tomography system (CT system) can be designed as a relatively narrow structure compared to typical prior art CT systems. This can be achieved in that a rotation of the detector and preferably also of the X-ray tube assembly is not necessary for a CT examination and therefore a less stable, lighter structure is possible. As a result, the gantry can also be implemented as a narrower structure. Furthermore, a precise alignment of the detector can be achieved relatively easily via the CT system according to an embodiment of the present invention. By providing detector segments it is furthermore possible to simplify a replacement of parts of the detector and a replacement of the entire detector can also be made easier owing to an achievable lower weight of the individual detector segments.

Disclosed herewith in particular is a computed tomography system comprising an examination region and a static gantry, wherein the gantry comprises a detector ring which encircles the examination region, wherein the detector ring comprises at least two detector segments, wherein the computed tomography system is configured in such a way that a position of the detector segments in an axial direction of the detector ring is adjustable, in particular their positions are adjustable relative to one another.

The term examination region is to be understood in a broad sense within the scope of this invention. The examination region is generally an area in which a subject or object or a part of a subject or object can be placed in order for it to be examined via the computed tomography system, in particular in order to collect image data thereof. The subject can be for example a human being, in particular a patient, or an animal. An object can be, for example, a part of a human being or animal, for example a limb, a torso, an organ, etc. However, an object can also be any other object, for example an entity of some sort.

The gantry is preferably arranged around the examination region. The term static gantry is to be understood in particular in contrast to a rotating gantry according to the prior art. The term static relates in this context to the use for a CT measurement. The gantry is in particular configured in such a way that it does not have to be rotated for a CT measurement or that it is not provided that the gantry will rotate substantially around the examination region during the CT measurement. Furthermore, it is not ruled out that the static gantry may also be generally rotatable, for example in the non-installed state or during the installation of the gantry.

The gantry comprises the detector ring. This relationship and the term gantry itself are to be understood in a broad sense. For example, it can be provided that the detector ring is secured to or mounted on a basic framework of the gantry. The detector ring is preferably a static detector ring corresponding to the static gantry. In particular, the detector ring is configured not to rotate around the examination region during a CT measurement. This has the advantage that fewer rotational forces occur, thereby enabling the gantry as a whole to be implemented in a less solid design. Material can be saved as a result and the CT system can be lighter overall. This enables the potential applications of the CT system to be extended where appropriate since less space and/or a less stable installation site can be provided. The word “ring” in the term “detector ring” is to be interpreted in a broad sense. For example, the detector ring may have an extension in the radial direction. The detector ring may be a 360° detector ring. Accordingly, the detector ring extends around the entire circumference of a circle. This is advantageous in that X-ray signals can consequently be captured in all radial directions. Alternatively, the detector ring may also be a subring. A subring does not extend around the entire circumference of a circle. This can be advantageous in order for example to provide a particularly lightweight or particularly affordable CT system. The detector ring encircles the examination region, that is to say in particular that the detector ring is arranged around the examination region.

The detector ring comprises at least two detector segments, preferably at least three detector segments. Each of the detector segments can preferably comprise one or more detector elements which are configured to detect X-rays. The subdivision of the detector ring into detector segments can advantageously simplify a handling of the detector ring, for example in that fewer large parts need to be moved during installation. Costs, for example for transportation, can also be reduced as a result. Furthermore, a modularization can be realized by the subdivision into detector segments. For example, individual detector segments can be replaced. In this respect the use of detector segments is already advantageous even without the adjustment capability in the axial direction. For example, the detector ring may comprise precisely three detector segments. Three detector segments can be particularly favorable both for a relatively simple installation on account of the lower weight and for an easy orientation owing to the achieved flexibility. It has been shown that three detector segments on the one hand allow a flexible adjustment of the orientation of the detector segments, yet on the other hand also permit a good stability and organization of the individual parts thanks to the use of not too many segments. However, it is also possible in principle for the detector ring to comprise more than three detector segments or precisely two detector segments.

A position of the detector segments is adjustable in an axial direction of the detector ring. The axial direction is in particular the direction that extends perpendicularly through an area of a circle or subarea of a circle formed by the detector ring. The axial direction can correspond to a z-direction of the computed tomography system. For example, the position of the individual detector segments can be adjusted individually in the axial direction. Advantageously, the position of the detector ring as a whole as well as with reference to the individual detector segments can therefore be adjustable. An adjustment of the whole detector ring can be useful in order to adjust the position in relation to one or more X-ray sources. It can be achievable in this way for example that the detector ring lies in the focal spot of the X-ray source(s). By an adjustment of the individual segments it is also advantageously possible in this case to compensate for irregularities in the structure of the gantry, of the X-ray source(s) or of the detector ring. Optionally, an orientation of the detector segments can also be adjustable. This enables an even more precise adjustment of the detector ring to be made. The computed tomography system can preferably be configured such that the detector segments can be fixed in place following the adjustment of the axial position.

The computed tomography system can preferably comprise a static X-ray source in addition. In particular, the X-ray source can likewise be ring-shaped and be arranged around the examination region. The X-ray source can comprise a plurality of X-ray generators distributed along the ring shape. The detector ring and the X-ray source can have a common axial direction. The ring-shaped X-ray source can preferably have a greater diameter than the detector ring such that, viewed in the radial direction, the X-ray source is arranged further outward around the examination region than the detector ring. The detector ring can preferably be arranged offset relative to the X-ray source in the axial direction. With such a configuration, a particularly small offset of detector ring to X-ray source can be possible, as a result of which the image quality can be improved (compared to a greater offset).

According to an embodiment variant, the detector segments are substantially ring segments, each of which embodies a subring of the detector ring. Ring segments have proved to be particularly practical for handling during installation, replacement and maintenance.

According to an embodiment variant, the detector segments each comprise a baseplate and at least one functional unit comprising detector elements which is fixedly mounted or mountable on the baseplate and removable from the baseplate. The baseplate can be substantially subring-shaped. Viewed in the axial direction, the baseplate can, by virtue of its own shape, define the external shape of the detector segments. Advantageously, a particularly good modular and flexible embodiment of the detector ring can be realized via the baseplate. The term baseplate is to be understood in a broad sense in this context. In particular, the baseplate does not have to be flat but can have a structured surface. For example, the baseplate can have a receiving device, securing device, means for receiving and/or securing the functional units. Advantageously, it can be made possible via the baseplate for individual functional units containing detector elements to be removed, for example for maintenance or replacement, without changing the general orientation of the detector segment since the baseplate remains in the installed and oriented state.

According to an embodiment variant, the computed tomography system comprises guide rails which extend substantially in the axial direction and by which a displacement of the detector segments in the axial direction can be guided. For example, the detector segments can be inserted and/or be insertable onto the guide rails. Optionally, the detector segments can each comprise one or more bushings through which the guide rails are guided and/or can be guided. Optionally, the detector segments can be seated on the guide rails and/or be placeable thereon. The guide rails can facilitate a displacement of the detector segments. For example, a manual displacement with little application of force can be made possible via the guide rails. The guide rails can be detachably secured or securable on the gantry. Advantageously, the guide rails can therefore be used for example for an installation or maintenance operation and then removed in order to prevent the guide rails from obstructing in normal operation. Alternatively, the guide rails can be provided permanently on the computed tomography system, in particular on the gantry. Advantageously, a permanent presence of the guide rails can speed up for example a maintenance or replacement of the detector segments since there is no need to mount the guide rails first.

According to an embodiment variant, replaceable spacers are provided between the detector segments and the gantry or a basic framework of the gantry so that the position of the detector segments in the axial direction of the detector ring can be adjusted via the spacers. The term spacers is to be understood in a broad sense within the scope of this invention. Spacers can describe any mechanism and/or means suitable for ensuring a clearance between a basic framework of the gantry and the respective detector segment. For example, the spacers can be equalizing shim washers. The spacers can be shim plates, for example. Shim plates can also be defined as leveling plates and/or adjusting plates. Advantageously, the spacers can regulate the distance between a focal spot of the X-ray source and the scan planes of the detector ring or of the detector elements in the axial direction. For example, it can be provided to release the detector ring or individual detector segments and to move them on the guide rails, as described herein, in the axial direction. Thereafter, a new spacer can be inserted on the gantry and the detector ring or the detector segment can be moved back again in the axial direction until the detector ring or the detector segment comes into contact with the spacer. An adjustment of the detector ring can therefore be completed particularly precisely and quickly. Additional manual adjustment steps that would otherwise be necessary can therefore be dispensed with. The spacers accordingly constitute a particularly inexpensive mechanism and/or means to facilitate an adjustment of the detector segments in the axial direction. Optionally, it can be provided to use the spacers, in particular the shim plates, in order to perform a tensioning of at least one of the detector segments, in particular at least one of the baseplates, in order to equalize a shape of the respective detector segment. This can be advantageous in particular in the case of warping caused by heat.

According to an embodiment variant, the spacers are made of a material containing metal, in particular steel. The properties of metal can be particularly well-suited for use as spacers. For example, heat dissipation from the detector, for example in the case of heat buildup due to operating conditions, can be improved by way of metals. Steel in particular can provide a good combination of flexibility in production and stability. Optionally, however, other materials are also conceivable for the spacers.

According to an embodiment variant, the spacers are elongate equalizing shim washers. An orientation of the spacers is preferably provided such that the longitudinal extension of the equalizing shim washers substantially corresponds to a radial direction. By orienting the longitudinal extension of the spacers in the radial direction it can be rendered possible to change or if necessary correct the planarity of the detector ring particularly effectively.

According to an embodiment variant, the gantry comprises a fastening means and/or fastener (also referred to as a fastening device) for fixing the spacers in place. Fastening means can enable the installation of the spacers to be made easier and/or more reliable. The fastening means can comprise screw holes, for example. The fastening means can comprise for example slots for inserting the spacers. The fastening means can comprise for example a click-&-release mechanism. The gantry can comprise multiple fastening means for multiple spacers per detector segment.

According to an embodiment variant, at least two spacers are provided for each of the detector segments. Advantageously, a plurality of spacers can enable a particularly good adjustment also of the orientation of the detector segments. Particularly advantageously, three to five equalizing shim washers, preferably four equalizing shim washers, can be provided per detector segment. This number of equalizing shim washers can likewise enable a stable installation of the detector segments, a good variability in the orientation of the detector segments, and a manageable amount of effort when fitting the equalizing shim washers.

According to an embodiment variant, the computed tomography system comprises a set of spacers having different thicknesses. With a set of spacers, an axial position of the detector ring or of the detector segments can be set or changed relatively quickly and easily. Changing the axial position can be accomplished for example simply by replacing or adding spacers.

The spacers can be provided for example in gradations of 0.01 to 0.5 mm, preferably 0.05 to 0.2 mm. Gradations in this range can be particularly useful for adjusting the position of the detector ring since on the one hand a precise adjustment is possible, yet on the other hand also not too many spacers are required.

According to an embodiment variant, the computed tomography system comprises at least one motor which is configured to adjust the position of at least one of the detector segments in the axial direction. The computed tomography system may comprise a plurality of motors, each of which is configured to adjust the position of at least one of the detector segments in the axial direction. The at least one motor, optionally a plurality of motors, can be embodied to adjust the position of a plurality, preferably all, of the detector segments in the axial direction. Advantageously, an automatic readjustment of the axial position of the detector segments can be realized via a motor. In this case the detector segments can be mounted on a guide as described herein and/or be guidable on the guide. Advantageously, the motor can be configured to enable a substantially stepless adjustment of the axial position. “Substantially stepless” is to be understood in this context within the scope of the precision possible in view of construction factors and due to other constraints (such as, for example, a reasonable cost-benefit outlay with which a certain precision can be achieved). The motor can be provided without the use of spacers. Optionally, the spacers can be combined with the motor, as described herein. The spacers can for example enable an additional positional stability, while the motor can simultaneously provide an automatic displacement of the detector ring. The motor can be a quick and easy-to-operate solution for moving the detector segments.

According to an embodiment variant, the motor is a linear drive and/or a spindle drive. The guide may optionally have a thread. The motor may comprise a shaft which drives the thread such that a displacement in the axial direction of the detector segment can be made possible by turning the thread. The motor can be arranged on the detector ring and/or on the detector segment. The linear drive can comprise a linear unit and a servomotor, the linear unit being driven by way of the servomotor. The linear unit can be the guide or correspond to the guide. The detector segment can be arranged directly on the linear unit.

According to an embodiment variant, the computed tomography system is configured to detect, via a system of sensors, deviations from a position of the detector ring rated as ideal. Advantageously, it can be determined automatically via the sensor system whether a position of the detector ring or of the detector segments should be adjusted. The sensor system can particularly advantageously be combined with the motor. This enables a fully automatic displacement and adjustment of the axial position of the detector ring or of the detector segments to be realized. The sensor system can for example comprise one or more distance sensors. At least one distance sensor can for example be affixed to at least one detector segment and/or to a basic framework of the gantry. The at least one distance sensor can be embodied to determine a distance between the detector segment and the basic framework of the gantry. The sensor system used can be the measurement sensor system of the computed tomography system itself. For example, the computed tomography system can be configured to detect, on the basis of measurement data, in particular on the basis of CT image data and/or X-ray image data, a deviation from the position of the detector ring rated as ideal. If the detector ring is not in the focus of the X-ray source, i.e. in particular is offset in the axial direction, this usually leads to a deterioration in image quality. The computed tomography system can be configured to deduce on the basis of the image quality of a computed tomography measurement or an X-ray measurement whether a deviation from the position of the detector ring rated as ideal is present. Advantageously, via the measurement on the basis of CT data, a position determination can be possible without requiring additional sensors. For example, it can be provided to determine the CT data in the course of a test measurement. Optionally, the computed tomography system can be configured to check, on the basis of CT data obtained during routine operation, for a deviation of the position of the detector ring or of the detector segments. The check can also be delegated to an external monitoring unit.

According to an embodiment variant, the computed tomography system comprises an X-ray source and a collimator for the X-ray source, the computed tomography system being configured in such a way that a position of the collimator in an axial direction of the detector ring is adjustable. The collimator may in particular comprise collimator segments. The adjustability of the collimator and/or the collimator segments can be provided analogously as described in relation to the adjustability of the detector ring or of the detector segments. For example, collimator spacers can be provided for adjusting an axial position of the collimator. The collimator spacers can be implemented analogously with properties as described in relation to the spacers of the detector segments. Advantageously, therefore, a position of the collimator can be just as adjustable as a position of the detector ring. In principle, a computed tomography system is also conceivable which comprises the adjustable collimator without having a detector ring as described herein. That is to say, the adjustable collimator can be realizable independently of the adjustable detector ring, and vice versa.

A further aspect of an embodiment of the present invention is a method for orienting a detector ring in or on a gantry of a computed tomography system, wherein the detector ring comprises at least two detector segments, wherein the gantry comprises an X-ray source, wherein the method comprises the following steps:
(a) determining measurement values which indicate a current position of the detector segments,
(b) establishing whether a deviation in the axial direction is present according to which the current position of the detector segments deviates from an ideal position,
(c) adjusting the position, in the axial direction, of the detector segments for which a deviation has been identified, in particular relative to the X-ray source and/or relative to one another.

All the advantages and features of the computed tomography system can be applied analogously to the method, and vice versa.

The measurement values can be determined via a sensor system. For example, the measurement values can be determined via at least one distance sensor. According to an embodiment variant, distance sensors are used on the gantry and the detector segments in order to obtain measurement values in the form of distance measurement data, the position being established on the basis of the distance measurement data. This can represent a simple mechanism and/or means for directly determining a position.

According to an embodiment variant, the deviation is identified by performing an X-ray scan, in particular a computed tomography scan, in order to obtain measurement values in the form of at least one X-ray image, and by establishing whether image errors occur in the at least one X-ray image which can be traced back to a position of the detector rings. The measurement values can be CT measurement values and/or X-ray data measured via the computed tomography system. The CT measurement values and/or X-ray data can indicate a current position based on the fact that a deviation from a focus position of the detector segments can lead to a deterioration in image quality. A deviation of the position in the axial direction of the detector segments can be identified on the basis of properties of the CT measurement values and/or X-ray data, for example a reduced image quality.

Advantageously, therefore, identifying the deviation can be possible without the use of an additional system of sensors. It can further be advantageous that the effects of the position are taken into account directly. Accordingly, this variant is not dependent on a position rated as ideal actually being ideal, but a position optimized or improved within the scope of the applied precision can be found directly on the basis of the image quality.

The adjustment of the position can be performed manually by a user. The user can be prompted by the computed tomography system via a notification to perform an adjustment of the position. Alternatively, the adjustment of the position can be initiated automatically by the computed tomography system.

According to an embodiment variant, the detector segments are moved in the axial direction on at least one guide rail in each case in order to adjust the position. Optionally, the at least one guide rail in each case can be mounted on the gantry prior to the adjustment. The at least one guide rail can be removed again after the adjustment. Alternatively, the guide rail can be permanently mounted on the gantry. The moving of the detector segments can be provided manually by a user. Alternatively, the moving of the detector segments can be provided automatically, in particular via a motor. Before the position is adjusted, at least one fastening means of the detector segments which are being adjusted can be released. The fastening means can in each case secure at least one detector segment on the gantry, in particular on a basic framework of the gantry. For example, the fastening means can be a fastening means as described herein. The fastening means can be secured in place again following the adjustment of the position.

According to an embodiment variant, spacers are used between the gantry and the detector segments in order to compensate for deviations of the detector segments. The spacers can be spacers as described herein. Deviations can be compensated for by selection from a set of different spacers. For example, a first spacer used for compensation can be replaced by another spacer different from the first. The spacers can be different in particular with regard to their thickness. Optionally, a position and/or orientation of the detector segments relative to one another can be balanced out via the spacers. Optionally, the detector segment, in particular the baseplate, can be tensioned via the spacers and a screwed connection of at least one detector segment, in particular a baseplate of the at least one detector segment. A tensioning can be carried out in order to compensate for an unevenness of the detector segment, in particular of the baseplate. This can be advantageous for example in the event of warping due to heat. The baseplate can be a baseplate as described herein.

According to an embodiment variant, the spacers are fixed in place via at least one fastener and/or fastening means, in particular one or more screws. The spacers can be fixed in place independently of the fixing of the detector segments. An installation can be simplified in this way since the spacers can be securely fixed in place when the detector segment is moved. Alternatively, the spacers can each be fixed in place together with the respective detector segment by way of at least one common fastening means. Advantageously, fewer fastening means may be required as a result.

According to an embodiment variant, the detector segments are offset in the axial direction via a motor in order to adjust the position. The motor can be a linear drive and/or a spindle drive, for example. Advantageously, this enables the position to be adjusted automatically without a user having to move the detector segments manually. A substantially stepless adjustment of the axial position can be provided via the motor.

According to an embodiment of the present invention, the computed tomography system may be configured such that a position of a scan plane of one detector segment of the at least two detector segments relative to a scan plane of another detector segment of the at least two detector segments in the axial direction of the detector ring is adjustable, in particular by moving the scan plane of the one detector segment of the at least two detector segments relative to the scan plane of the other detector segment of the at least two detector segments in the axial direction of the detector ring. For each detector segment of the at least two detector segments the scan plane of that detector segment may be perpendicular to the axial direction. For each detector segment of the at least two detector segments the scan plane of that detector segment may extend through the detector elements of the at least one functional unit of that detector segment.

According to an embodiment of the present invention, the computed tomography system may be configured such that a position of a baseplate of one detector segment of the at least two detector segments relative to a baseplate of another detector segment of the at least two detector segments in the axial direction of the detector ring is adjustable, in particular by moving the baseplate of the one detector segment of the at least two detector segments relative to the baseplate of the other detector segment of the at least two detector segments in the axial direction of the detector ring. For each detector segment of the at least two detector segments the baseplate of that detector segment may be perpendicular to the axial direction. For each detector segment of the at least two detector segments the baseplate of that detector segment may be parallel to the scan plane of that detector segment.

Unless explicitly stated otherwise, all the embodiment variants described herein can be combined with one another.

### BRIEF DESCRIPTION OF THE DRAWINGS

Embodiment variants are described below with reference to the attached figures.

FIG. 1 shows a computed tomography system according to an embodiment variant of the present invention,

FIG. 2 shows a basic framework of a gantry of a computed tomography system according to an embodiment variant of the present invention,

FIG. 3 shows an exploded view of a gantry of a computed tomography system according to an embodiment variant of the present invention,

FIG. 4 shows a gantry of a computed tomography system according to an embodiment variant of the present invention in which two out of three detector segments are installed,

FIG. 5 shows a corresponding gantry of a computed tomography system according to the same embodiment variant of the present invention as shown in FIG. 4 in which all three detector segments of the detector ring are installed,

FIG. 6 shows a gantry of a computed tomography system according to an embodiment variant of the present invention during the installation of a detector segment that has been moved away from the gantry,

FIG. 7 shows a gantry of a computed tomography system according to an embodiment variant of the present invention during the installation of a detector segment that has been moved toward the gantry, and

FIG. 8 shows a flowchart of a method for orienting a detector ring in or on a gantry of a computed tomography system according to an embodiment variant of the present invention.

### DETAILED DESCRIPTION

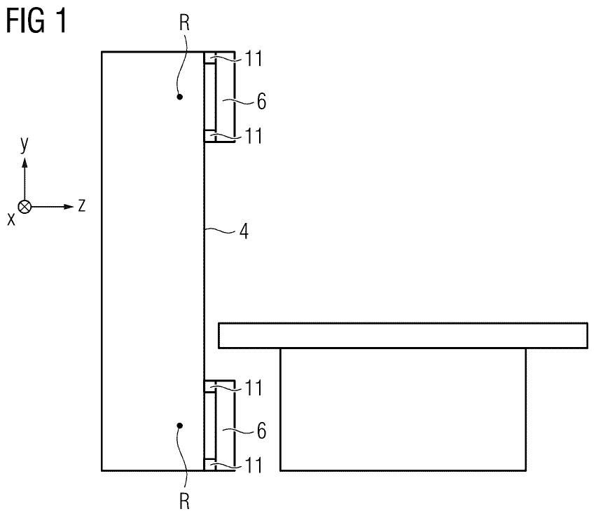

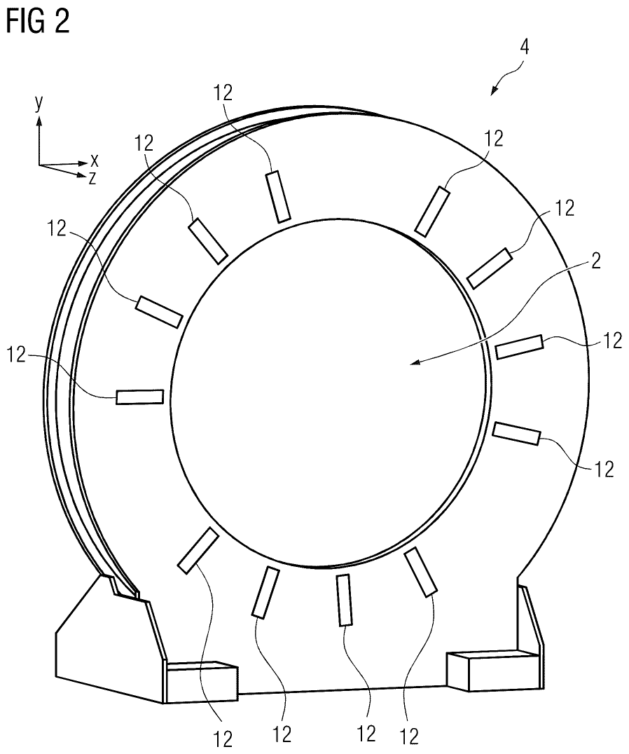

FIG. 1 shows a computed tomography system according to an embodiment variant of the present invention. The computed tomography system comprises a static gantry 4 having a detector ring 6 which encircles an examination region 2 (not directly visible here due to the perspective, cf. examination region 2 in FIG. 2). The detector ring 6 comprises at least two detector segments 7, which cannot be seen here due to the perspective, though they can be seen in some of the following embodiment variants shown. The computed tomography system is configured in such a way that a positioning of the detector segments 7 in an axial direction, which in this case corresponds to the z-direction, of the detector ring 6 can be adjusted. Replaceable spacers 11 are provided between the detector segments 7 and the gantry 4 so that the positioning of the detector segments 7, which when combined with one another produce the detector ring 6, in the axial direction (z-direction) of the detector ring 6 can be adjusted via the spacers 11. Viewed in the z-direction opposite the detector ring 6, on the other side of the gantry 4, an X-ray source R and a collimator for the X-ray source R are provided. Optionally, the computed tomography system can be further configured in such a way that a positioning of the collimator in an axial direction (z-direction) of the detector ring 6 can be adjusted.

FIG. 2 shows a basic framework of a gantry 4 according to an embodiment variant of the present invention. The gantry 4 comprises fastening means 12 (also referred to as fastener 12), in this case screw holes and slots, for fixing the spacers 11 in place. Alternatively, other fastening means 12, for example a click-&-release mechanism, can also be provided. Provided centrally in the gantry 4 is an examination region 2 in which for example a patient can be placed.

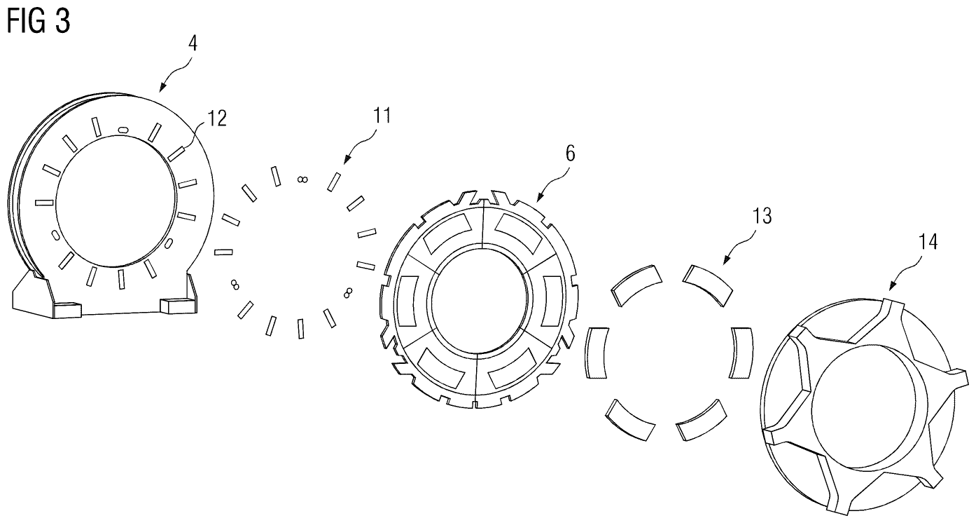

FIG. 3 shows an exploded view of a gantry 4 of a computed tomography system according to an embodiment variant of the present invention. The basic framework of the gantry 4 substantially corresponds to the gantry 4 shown in FIG. 2. Additionally provided here are spacers 11, a detector ring 6 and electronics modules 13 for the detector elements of the detector segments 7. The spacers 11 can preferably be made out of a material containing metal, in particular steel. For example, the spacers 11 can be plates which can be fixed in place on the fastening means 12 via screws. In this embodiment variant, the spacers 11 are elongate equalizing shim washers whose longitudinal extension runs substantially in the radial direction. A set of spacers 11 having different thicknesses can be provided, each of which can be replaced as needed. A gradation of the thickness can amount to 0.1 mm, for example. During operation, the detector ring 6 can be covered externally via a cover panel 14.

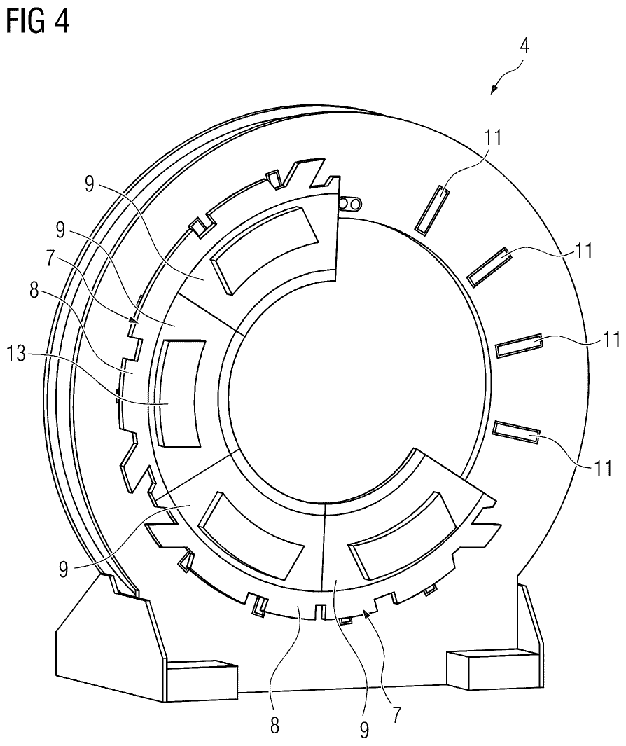

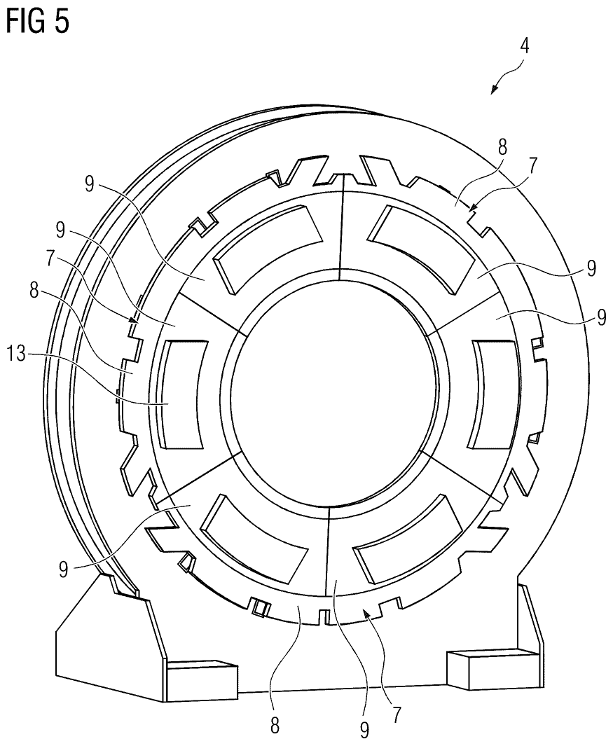

FIG. 4 shows a gantry 4 of a computed tomography system according to an embodiment variant of the present invention in which two out of three detector segments 7 are installed. FIG. 5 shows a corresponding gantry 4 of a computed tomography system according to the same embodiment variant of the present invention in which all three detector segments 7 of the detector ring 6 are installed. To provide a better view, a cover panel 14 is omitted in this illustration. The detector segments 7 are substantially ring segments, each of which forms a subring of the detector ring 6. Each of the detector segments 7 comprises a respective baseplate 8 and two each of functional units 9 containing detector elements secured to the baseplate 8 and removable from the baseplate 8. Also provided in each case on each functional unit 9 is an electronics module 13 for controlling and reading out the detector elements. The baseplates 8 are also embodied substantially in a subring shape. In this embodiment variant, four spacers 11 are provided in each case for each of the detector segments 7 or for each baseplate 8.

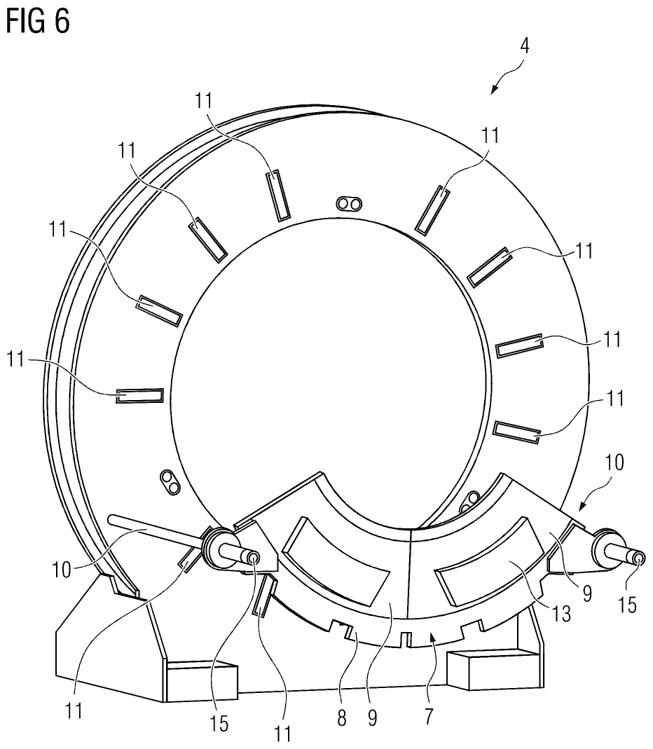

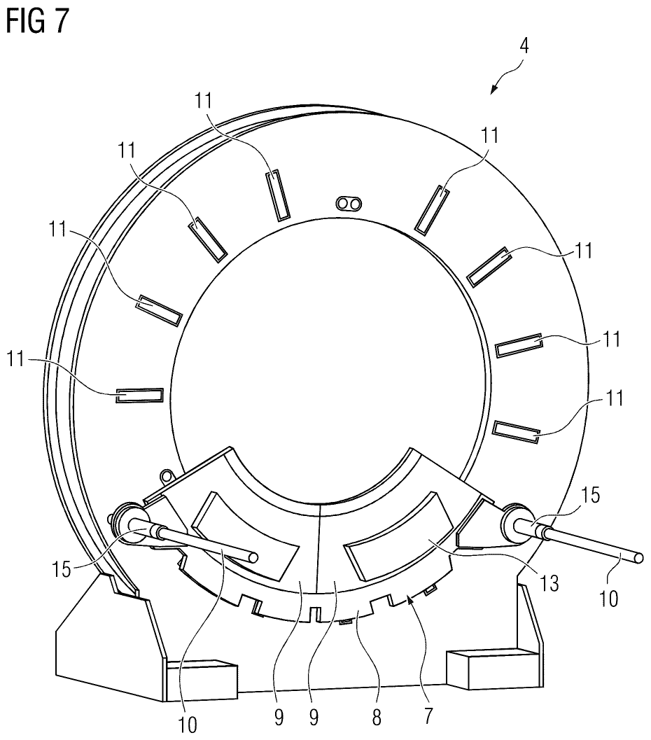

FIGS. 6 and 7 each show a gantry 4 of a computed tomography system according to an embodiment variant of the present invention during the installation or repositioning of a detector segment 7. In FIG. 7, the detector segment 7 is moved toward the gantry 4 and a positioning of the detector segment 7 is determined via the spacers 11 between gantry 4 and detector segment 7. To be seen in particular are spacers 11 which are provided for further detector segments 7. In FIG. 6, the detector segment 7 has been moved away from the gantry 4 on guide rails 10 so that the spacers 11 of the detector segment 7 can be replaced in order to adjust a positioning of the detector segment 7 in an axial direction of the detector ring 6. The guide rails 10 extend substantially in the axial direction and serve to guide the repositioning of the detector segment 7. Optionally, the computed tomography system can comprise motors (placed for example at 15) which are configured to adjust the positioning at least of the detector segment 7 in the axial direction. The motor can be a spindle drive and the guide rail 10 can be a threaded rod that is used for a movement caused by the motor. The guide rails 10 can be removable. If the guide rails 10 are provided as not removable, which can be advantageous in particular in the version with motor, then it is advantageous to make the guide rails 10 somewhat shorter in order to avoid the guide rails 10 protruding. In the version with motor, the spacers 11 can also be omitted and the positioning of the detector segment 7 can be adjusted solely by way of the motor.

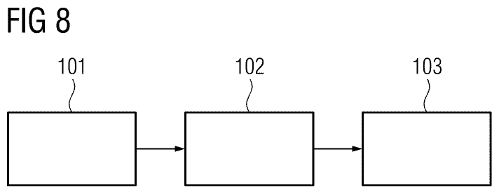

FIG. 8 shows a flowchart of a method for orienting a detector ring 6 in or on a gantry 4 of a computed tomography system according to an embodiment variant of the present invention. The detector ring 6 in this case comprises at least two detector segments 7. In a first step 101 of the method, measurement values are determined which indicate a current positioning of the detector segments 7. The measurement values can be determined for example via a sensor system. The sensor system can for example comprise one or more distance sensors which determine distance measurement data.

Alternatively or in addition, the CT measurement sensor system itself can be used by performing an X-ray scan, in particular a computed tomography scan, in order to obtain measurement values in the form of at least one X-ray image and/or CT data. In a further step 102, it is established whether a deviation in the axial direction is present according to which the current positioning of the detector segments 7 deviates from an ideal positioning. The positioning or the deviation can be ascertained for example on the basis of the distance measurement data determined via the distance sensors. Alternatively or in addition, the deviation can be identified by establishing whether image errors are present in the measured X-ray image and/or the measured CT data which can be traced back to a positioning of the detector rings 6. In a further step 103, if a deviation has been identified, the positioning of one or more detector segments 7 in the axial direction of the detector segments 7 is adjusted. In order to adjust the positioning, the detector segments 7 can optionally be moved in the axial direction on guide rails 10. In addition or alternatively, in order to adjust the positioning, the detector segments 7 can be moved in the axial direction via a motor.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of the embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing embodiments. The present invention may, however, be embodied in many alternate forms and should not be construed as limited to only the embodiments set forth herein.

What is claimed is:

1. A computed tomography system comprising:
an examination area; and
a static gantry including an X-ray source and a detector ring, wherein
the detector ring encircles the examination area and includes at least two detector segments, and
the computed tomography system is configured such that a position of the at least two detector segments relative to one another in an axial direction of the detector ring is adjustable.

2. The computed tomography system as claimed in claim 1, wherein each of the at least two detector segments comprises:
a baseplate; and
at least one functional unit containing detector elements, the at least one functional unit configured to be removably secured to the baseplate.

3. The computed tomography system as claimed in claim 1, further comprising:
guide rails extending substantially in the axial direction, the guide rails configured to guide a displacement of the at least two detector segments in the axial direction.

4. The computed tomography system as claimed in claim 1, further comprising:
replaceable spacers arranged between the at least two detector segments and the static gantry such that the position of the at least two detector segments in the axial direction of the detector ring is adjustable via the replaceable spacers.

5. The computed tomography system as claimed in claim 4, wherein the static gantry comprises:
at least one fastener configured to fix the replaceable spacers in place.

6. The computed tomography system as claimed in claim 4, wherein at least two replaceable spacers are provided for each of the at least two detector segments.

7. The computed tomography system as claimed in claim 1, further comprising:
at least one motor configured to adjust a position of at least one of the at least two detector segments in the axial direction.

8. The computed tomography system as claimed in claim 7, wherein the at least one motor includes at least one of a linear drive or a spindle drive.

9. The computed tomography system as claimed in claim 1, wherein the computed tomography system is configured to detect, via a sensor system, deviations from an ideal position of the detector ring.

10. The computed tomography system as claimed in claim 1, further comprising:
a collimator for the X-ray source, wherein
the computed tomography system is configured such that a position of the collimator in the axial direction of the detector ring relative to the X-ray source is adjustable.

11. A method for orienting a detector ring in or on a gantry of a computed tomography system, wherein the detector ring includes at least two detector segments, and wherein the method comprises:
determining measurement values indicating a current position of the at least two detector segments of the detector ring;
establishing whether a deviation in an axial direction is present, the deviation being a deviation by which the current position of the at least two detector segments deviates from an ideal position; and
adjusting a position of the at least two detector segments in the axial direction in response to establishing the deviation from the ideal position.

12. The method as claimed in claim 11, wherein spacers are used between the gantry and the at least two detector segments to compensate for deviations of the at least two detector segments.

13. The method as claimed in claim 11, further comprising:
adjusting the position of the at least two detector segments by moving the at least two detector segments in the axial direction via a motor.

14. The method as claimed in claim 11, further comprising:
identifying the deviation by performing an X-ray scan to obtain the measurement values in the form of at least one X-ray image, and
establishing whether image errors, which are traceable back to the position of the at least two detector segments, occur in the at least one X-ray image.

15. The method as claimed in claim 11, further comprising:
using distance sensors on the gantry and the at least two detector segments to obtain the measurement values in the form of distance measurement data, and
ascertaining the current position based on the distance measurement data.

16. The method as claimed in claim 14, further comprising:
using distance sensors on the gantry and the at least two detector segments to obtain the measurement values in the form of distance measurement data, and
ascertaining the current position based on the distance measurement data.

17. The computed tomography system as claimed in claim 2, further comprising:
guide rails extending substantially in the axial direction, the guide rails configured to guide a displacement of the at least two detector segments in the axial direction.

18. The computed tomography system as claimed in claim 3, further comprising:
replaceable spacers arranged between the at least two detector segments and the static gantry such that the position of the at least two detector segments in the axial direction of the detector ring is adjustable via the replaceable spacers.

19. The computed tomography system as claimed in claim 3, wherein the computed tomography system is configured to detect, via a sensor system, deviations from an ideal position of the detector ring.

20. The computed tomography system as claimed in claim 3, further comprising:
a collimator for the X-ray source, wherein
the computed tomography system is configured such that a position of the collimator in the axial direction of the detector ring relative to the X-ray source is adjustable.
